SEQUENCE LISTING (3 sequences)

SEQ ID NO: 1 - DNA, 65 bp, synthetic construct
  source 1..65: mol_type = other DNA
ggttggtgtggttgggggttagggttagggttagggagtccgtggtagggcaggttggggtgact

SEQ ID NO: 2 - DNA, 15 bp, synthetic construct
  source 1..15: mol_type = other DNA
ggttggtgtggttgg

SEQ ID NO: 3 - DNA, 29 bp, synthetic construct
  source 1..29: mol_type = other DNA
agtccgtggtagggcaggttggggtgact

(19) United States
(12) Patent Application Publication
(10) Pub. No.: US 2025/0041479 A1
(43) Pub. Date: Feb. 6, 2025

(54) PHOTO-ENZYMATIC PRINTING

(71) Applicant: The Board of Trustees of the Leland Stanford Junior University, Stanford, CA (US)

(72) Inventors: Joshua Sampson, Stanford, CA (US); Mark Skylar-Scott, Palo Alto, CA (US); Steven G. Boxer, Stanford, CA (US); Zhe Ji, Stanford, CA (US); Elisa Marani, Stanford, CA (US)

(21) Appl. No.: 18/715,480

(22) PCT Filed: Dec. 3, 2022
(86) PCT No.: PCT/US2022/080871

§ 371 Date: May 31, 2024

Related U.S. Application Data
(60) Provisional application No. 63/285,921, filed on Dec. 3, 2021.

(51) Int. Cl. A61L 27/22; A61L 27/38; A61L 27/50; B33Y 10/00

(52) U.S. Cl. CPC A61L 27/225; A61L 27/3808; A61L 27/50; B33Y 10/00

(57) ABSTRACT

Methods of making fibrin mesh are described.

### PRIORITY CLAIM

This application claims benefit to U.S. Provisional Application Ser. No. 63/285,921, filed Dec. 3, 2021, which is incorporated herein by reference in its entirety.

### BACKGROUND OF THE INVENTION

While light-based 3D bioprinters can manufacture cell-laden tissue-like structures with superlative geometric complexity, existing methods rely on free radical chemistry to crosslink synthetic acrylate or thiolene photopolymers (Bishop et al. (2017). Genes Dis. 4 (4): 185 PMC6003668), generating materials with poor degradability and biocompatibility. Conversely, natural extracellular matrix (ECM) scaffolds, such as fibrin, have been used to create clinically relevant Tissue Engineerecd Vascular Grafts (TEVGs) with high burst pressures and long-term stability (Niklason et al. (2020) Science 370 (6513)), however until now it was not possible to photo-print these natural ECMs.

Methods of photo-printing natural scaffolds would be an advancement in the art.

### SUMMARY OF THE INVENTION

In a first aspect, the invention provides a method of making a fibrin mesh, comprising: a) administering a pattern of light of a first wavelength into a solution, wherein the solution comprises a first biopolymer subunit and a photoswitchable thrombin-like enzyme system, wherein the light of a first wavelength either activates or inhibits the thrombin-like enzyme in the system, and optionally administering a pattern of light of a second wavelength into the solution, wherein the light of a second wavelength that either activates or inhibits the thrombin-like enzyme in the system; b) repeating a) as necessary, thereby forming the fibrin mesh, wherein the fibrin mesh is not otherwise modified with photocrosslinkable moieties.

In another aspect, the invention provides a method of making a fibrin mesh, comprising: a) administering a pattern of light of a first wavelength into a solution, wherein the solution comprises a first biopolymer subunit and a photoswitchable thrombin-like enzyme system, wherein the first wavelength is between about 420 nm and about 500 nm, thereby activating the thrombin-like enzyme in the thrombin-like enzyme system; b) administering a pattern of light of a second wavelength into the solution, wherein the second wavelength is between about 320 nm and about 400 nm, thereby deactivating the thrombin-like enzyme in the thrombin-like enzyme system; c) repeating a) and b) as necessary, thereby forming the fibrin mesh, wherein the fibrin mesh is not otherwise modified with photocrosslinkable moieties wherein the first biopolymer subunit is fibrinogen, and the photoswitchable thrombin-like enzyme system comprises thrombin, a DNA inhibitor of thrombin, and a photoswitchable inhibitor of the DNA inhibitor of thrombin. In an exemplary embodiment, the method further comprises d) washing the product of step c) with a washing buffer.

In an exemplary embodiment, the first biopolymer subunit is fibrinogen. In an exemplary embodiment, the photoswitchable thrombin-like enzyme system comprises thrombin, a DNA inhibitor of thrombin (such as Itelo), and a photoswitchable inhibitor of the DNA inhibitor of thrombin (such as Razo).

In an exemplary embodiment, the solution further comprises mammalian cells. In an exemplary embodiment, the solution further comprises human cells. In an exemplary embodiment, the solution further comprises fibroblasts. In an exemplary embodiment, the solution further comprises mammalian fibroblasts. In an exemplary embodiment, the solution further comprises human fibroblasts.

In an exemplary embodiment, the method further comprises administering mammalian cells to the fibrin mesh. In an exemplary embodiment, the method further comprises administering human cells to the fibrin mesh. In an exemplary embodiment, the method further comprises administering fibroblasts to the fibrin mesh. In an exemplary embodiment, the method further comprises administering mammalian fibroblasts to the fibrin mesh. In an exemplary embodiment, the method further comprises administering human fibroblasts to the fibrin mesh.

### DESCRIPTION OF THE DRAWINGS

FIG. 1 shows the device.

FIG. 2 shows Razo-Itelo clotting kinetics.

FIG. 3 shows Razo cis-trans conversion using light.

FIG. 4 shows Razo cis-trans thermal conversion in the dark.

FIG. 5 shows Razo cis-trans interconversion durability.

FIG. 6 shows the inverted stereolithography setup.

FIG. 7 shows the Stanford logo printed as an opaque fibrin mesh.

FIG. 8A shows a maximum intensity z-projection of the fibrin mesh captured using fluorescence confocal microscopy. FIG. 8B further demonstrates the resolution of the fibrin mesh.

FIG. 9F shows that printed constructs with fibroblasts show high viability, as demonstrated by a high ratio of calceinAM (green):ethidium homodimer-1 (red) staining. FIG. 9G shows fibroblasts imaged by using phalloidin-Alexa Fluor 488 (to stain F-actin) and DAPI (to stain nuclei) to visualize cell morphology.

FIG. 10A shows perfused chips after washing, FIG. 10B shows perfused chips after 24 hours, media slowly diffused through the printed fibrin. FIG. 10C shows print immediately after printing. FIG. 10D shows print after printing and washing, in which PBS is perfused through to wash away unpolymerized fibrinogen.

FIG. 11A shows the schematic of the perfusion system installed inside an incubator A pump that delivers a continuous perfusion of media to the cell-embedded fibrin structure inside the PDMS chip. FIG. 11B is a picture of a perfusion setup.

FIG. 12 shows the channel observed at 100× magnification.

FIG. 13A shows HNDF cells (labeled with CellTracker Red, ThermoFisher) embedded in fibrin imaged 0 hours after printing. FIG. 13B shows HNDF cells embedded in fibrin imaged 24 hours after printing. FIG. 13C shows HNDF cells embedded in fibrin imaged 48 hours after printing.

### DETAILED DESCRIPTION OF THE INVENTION

### I. Introduction

In one aspect, the invention provides a method of making high resolution patterns of natural fibrin scaffolds. In an exemplary embodiment, the method utilizes a photoswitchable thrombin-like enzyme system that enables the photo-induced activation (between about 420 nm and about 500 nm, or between about 461 nm and about 489 nm, or about 475 nm) or inhibition (UV, between about 320 nm and about 400 nm, or between about 346 nm and about 374 nm, or about 360 nm) of a thrombin-like enzyme, an enzyme that catalyzes the cleavage of fibrinogen and subsequent formation of a fibrin scaffold (Tian et al. (2016) J Am Chem Soc 138 (3): 955). In this photoswitchable thrombin-like enzyme system, blue light exposure drives a cis-trans isomerization in an azobenzene derivative (Razo) that, in turn, inhibits an inhibitor (Itelo) of a thrombin-like enzyme. UV exposure drives a trans-cis isomerization of Razo that un-inhibits Itelo, which inhibits a thrombin-like enzyme. Thus, when a mixture of this photoswitchable thrombin-like enzyme system, fibrinogen, and optionally mammalian cells (such as fibroblasts) are exposed to patterns of blue light and UV light, the activated thrombin-like enzyme catalyzes the formation of a fibrin mesh. This invention makes possible the formation of fibrin mesh which are essentially free of gelatin methacryloyl or polyethylene glycol diacrylate. In an exemplary embodiment, between about 0.01% and about 5%, or between about 0.01% and about 4%, or between about 0.01% and about 3%, or between about 0.01% and about 3%, or between about 0.01% and about 2%, or between about 0.01% and about 1%, or between about 0.01% and about 0.5% of the volume of the fibrin mesh contains gelatin methacryloyl or polyethylene glycol diacrylate. In an exemplary embodiment, between about 0.01% and about 5%, or between about 0.01% and about 4%, or between about 0.01% and about 3%, or between about 0.01% and about 3%, or between about 0.01% and about 2%, or between about 0.01% and about 1%, or between about 0.01% and about 0.5% of the weight of the fibrin mesh contains gelatin methacryloyl or polyethylene glycol diacrylate.

### II. Methods

In another aspect, the invention provides a method of making a fibrin mesh, comprising: a) administering a pattern of light of a first wavelength into a solution, wherein the solution comprises a first biopolymer subunit and a photoswitchable thrombin-like enzyme system, wherein the light of a first wavelength results either in activating or inhibiting the thrombin-like enzyme in the photoswitchable thrombin-like enzyme system; b) administering a pattern of light of a second wavelength into the solution, wherein the light of a second wavelength results either in activating or inhibiting the thrombin-like enzyme in the photoswitchable thrombin-like enzyme system; c) repeating a) and b) as necessary, thereby forming the fibrin mesh. In an exemplary embodiment, the fibrin mesh is not otherwise modified with photocrosslinkable moieties. In an exemplary embodiment, the photoswitchable thrombin-like enzyme system comprises a thrombin-like enzyme, a DNA inhibitor of the thrombin-like enzyme, and a photoswitchable inhibitor of the DNA inhibitor of the thrombin-like enzyme. In an exemplary embodiment, when the light of a first wavelength activates the thrombin-like enzyme, then the light of a second wavelength inhibits the thrombin-like enzyme. In an exemplary embodiment, when the light of a first wavelength inhibits the thrombin-like enzyme, then the light of a second wavelength activates the thrombin-like enzyme. In an exemplary embodiment, the photocrosslinkable moieties are acrylates, epoxides, vinyl esters, and thiolenes. In an exemplary embodiment, the photocrosslinkable moieties are acrylates, epoxides, vinyl esters, or thiolenes. In an exemplary embodiment, the method further comprises d) washing the product of step c) with a washing buffer.

IIa. Fibrin Mesh

In an exemplary embodiment, the fibrin mesh does not contain mammalian cells. In an exemplary embodiment, the fibrin mesh is a mammalian cell-containing fibrin mesh. In an exemplary embodiment, the fibrin mesh is a fibroblast-containing fibrin mesh. In an exemplary embodiment, the fibrin mesh is a cardiomyocyte-containing fibrin mesh. In an exemplary embodiment, the fibrin mesh is an endothelial cell-containing fibrin mesh.

IIb. Pattern

In an exemplary embodiment, the pattern is a screen placed between the solution and the light source which prevents light from reaching certain portions of the solution. In an exemplary embodiment, the pattern is a screen placed between the solution and the light source which prevents light from reaching certain portions of the solution and allows light to reach certain portions of the solution. In an exemplary embodiment, a first pattern is provided for the light of a first wavelength described herein, and a second pattern is provided for the light of a second wavelength described herein.

IIc. Light of a First Wavelength

In an exemplary embodiment, the light of a first wavelength activates the thrombin-like enzyme of the photoswitchable thrombin-like enzyme system. In an exemplary embodiment, the light of a first wavelength is blue light. In an exemplary embodiment, the light of a first wavelength is between about 420 nm and about 500 nm. In an exemplary embodiment, the light of a first wavelength is between about 461 nm and about 489 nm. In an exemplary embodiment, the first wavelength is administered between about 0.1 seconds and about 20 seconds. In an exemplary embodiment, the first wavelength is administered between about 0.1 seconds and about 10 seconds. In an exemplary embodiment, the first wavelength is administered between about 0.5 seconds and about 5 seconds. In an exemplary embodiment, the solution receives a first wavelength intensity of between about 10 mW/cm² and about 30 mW/cm². In an exemplary embodiment, the solution receives a first wavelength intensity of between about 18.5 mW/cm² and about 22.5 mW/cm².

In an exemplary embodiment, the light of a first wavelength activates the thrombin-like enzyme in the photoswitchable thrombin-like enzyme system, and the light of a second wavelength inhibits the thrombin-like enzy me in the photoswitchable thrombin-like enzyme system.

IId. Solution

In an exemplary embodiment, the solution comprises a first biopolymer subunit and a photoswitchable thrombin-like enzyme system. In an exemplary embodiment, the solution further comprises a buffer. In an exemplary embodiment, the solution further comprises potassium. In an exemplary embodiment, the solution further comprises calcium. In an exemplary embodiment, the solution further comprises sodium. In an exemplary embodiment, the solution further comprises mammalian cells. In an exemplary embodiment, the solution is essentially free of photocrosslinkable moieties. In an exemplary embodiment, the solution is essentially free of synthetic photocrosslinkable moieties. In an exemplary embodiment, the photocrosslinkable moieties are acrylates, epoxides, vinyl esters, and/or thiolenes. In an exemplary embodiment, between about 0.001% and about 0.01%, between about 0.01% and about 5%, or between about 0.01% and about 4%, or between about 0.01% and about 3%, or between about 0.01% and about 3%, or between about 0.01% and about 2%, or between about 0.01% and about 1%, or between about 0.01% and about 0.5% of the volume of the solution contains photocrosslinkable moieties. In an exemplary embodiment, between about 0.001% and about 0.01%, between about 0.01% and about 5%, or between about 0.01% and about 4%, or between about 0.01% and about 3%, or between about 0.01% and about 3%, or between about 0.01% and about 2%, or between about 0.01% and about 1%, or between about 0.01% and about 0.5% of the weight of the solution contains synthetic photocrosslinkable moieties

### IId1. First Biopolymer Subunit

In an exemplary embodiment, the solution comprises a first biopolymer subunit. In an exemplary embodiment, the first biopolymer subunit is fibrinogen. Fibrinogen can be purchased through commercial suppliers such as Sigma-Aldrich. In an exemplary embodiment, the first biopolymer subunit is fibrinogen. In an exemplary embodiment, the first biopolymer subunit is fibrinogen. In an exemplary embodiment, the fibrinogen comprises an alpha chain, a beta chain, and a gamma chain. In an exemplary embodiment, the first biopolymer subunit is human fibrinogen. In an exemplary embodiment, the human fibrinogen comprises a human alpha chain, a human beta chain, and a human gamma chain. In an exemplary embodiment, the first biopolymer subunit is bovine fibrinogen. In an exemplary embodiment, the bovine fibrinogen comprises a bovine alpha chain, a bovine beta chain, and a bovine gamma chain. In an exemplary embodiment, prior to the administering of step a), the fibrinogen is present in the solution at a concentration of between about 3 mg/mL and about 100 mg/mL. In an exemplary embodiment, wherein, prior to the administering of step a), the fibrinogen is present in the solution at a concentration of between about 10 mg/mL and about 50 mg/mL. Representative human fibrinogen protein sequences can be found as follows: human fibrinogen alpha chain—Genbank Gene ID 2243; human fibrinogen beta chain—Genbank Gene ID 2244; and human fibrinogen gamma chain—Genbank Gene ID 2266. Representative bovine fibrinogen protein sequences can be found as follows: bovine fibrinogen alpha chain—Genbank Gene ID 522039: bovine fibrinogen beta chain—Genbank Gene ID 510522; and bovine fibrinogen gamma chain—Genbank Gene ID 280792. In an exemplary embodiment, the fibrinogen comprises an alpha chain with at least 90%, 91%, 92%, 93%, 94%, 95%, 96%, 97%, 98% or 99% identity with the sequence of Genbank Gene ID 2243. In an exemplary embodiment, the fibrinogen comprises an alpha chain with at least 90%, 91%, 92%, 93%, 94%, 95%, 96%, 97%, 98% or 99% identity with the sequence of Genbank Gene ID 522039. In an exemplary embodiment, the fibrinogen comprises a beta chain with at least 90%, 91%, 92%, 93%, 94%, 95%, 96%, 97%, 98% or 99% identity with the sequence of Genbank Gene ID 2244. In an exemplary embodiment, the fibrinogen comprises a beta chain with at least 90%, 91%, 92%, 93%, 94%, 95%, 96%, 97%, 98% or 99% identity with the sequence of Genbank Gene ID 510522. In an exemplary embodiment, the fibrinogen comprises a gamma chain with at least 90%, 91%, 92%, 93%, 94%, 95%, 96%, 97%, 98% or 99% identity with the sequence of Genbank Gene ID 2266. In an exemplary embodiment, the fibrinogen comprises a gamma chain with at least 90%, 91%, 92%, 93%, 94%, 95%, 96%, 97%, 98% or 99% identity with the sequence of Genbank Gene ID 280792. A fibrinogen-like protein is capable of being cleaved by thrombin to form fibrin that can subsequently form fibrils and/or a mesh. Fibrinogen-like protein activity can be ascertained from the light-scattering intensity (LSI) assay described in the Examples herein. In the assay, 100 U/ml thrombin and a 1 cm sample path length, addition of a fibrinogen-like protein at up to 200 mg/ml concentration results in at least a 10% reduction in transmitted light power within 6 hours. Fibrinogen is cleaved by a thrombin-like enzyme to form fibrin. The fibrin can polymerize into chains of fibrin which are called fibrils. Fibrils can associate with other fibrils, thus forming a fibrin mesh.

### IId2. Photoswitchable Thrombin-Like Enzyme System

In an exemplary embodiment, the solution comprises a photoswitchable thrombin-like enzyme system. In an exemplary embodiment, the photoswitchable thrombin-like enzyme system comprises a thrombin-like enzyme, a DNA inhibitor of the thrombin-like enzyme, and a photoswitchable inhibitor of the DNA inhibitor of the thrombin-like enzyme. In an exemplary embodiment, when the light of a first wavelength activates the thrombin-like enzyme, then the light of a second wavelength inhibits the thrombin-like enzyme. In an exemplary embodiment, when the light of a first wavelength inhibits the thrombin-like enzyme, then the light of a second wavelength activates the thrombin-like enzyme. In an exemplary embodiment, the photoswitchable thrombin-like enzyme system is an azobenzene or azobenzene derivative photoswitchable thrombin-like enzyme system. In an exemplary embodiment, the DNA inhibitor of the thrombin-like enzyme and the photoswitchable inhibitor of the DNA inhibitor of the thrombin-like enzyme are part of the same molecule (such as, for example, 7c6azo, which can be purchased from Stanford Protein and Nucleic Acid facility). In an exemplary embodiment, the DNA inhibitor of the thrombin-like enzyme and the photoswitchable inhibitor of the DNA inhibitor of the thrombin-like enzyme are separate molecules. In an exemplary embodiment, the thrombin-like enzyme is thrombin. In an exemplary embodiment, the DNA inhibitor of the thrombin-like enzyme is itelo. In an exemplary embodiment, the photoswitchable inhibitor of the DNA inhibitor of the thrombin-like enzyme is razo. In an exemplary embodiment, the thrombin-like enzyme is thrombin, the DNA inhibitor of the thrombin-like enzyme is itelo, and the photoswitchable inhibitor of the DNA inhibitor of the thrombin-like enzyme is razo.

Thrombin-like enzyme: A thrombin-like enzyme cleaves fibrinogen, thus forming fibrin that can subsequently form fibrils and/or a mesh. Examples of thrombin-like enzymes include thrombin and Snake Venom Thrombin-Like Enzymes (SVTLEs). In an exemplary embodiment, the thrombin-like enzyme is thrombin. In an exemplary embodiment, the thrombin-like enzyme is human thrombin. In an exemplary embodiment, the thrombin-like enzyme is bovine thrombin. In a light-scattering intensity (LSI) assay as described in the patent performed with 10 mg/ml fibrinogen and a 1 cm sample path length, addition of a thrombin-like enzyme at up to 1 mM concentration results in at least a 10% reduction in transmitted light power within 6 hours. Thrombin can be purchased through commercial suppliers such as Sigma-Aldrich. Representative thrombin protein sequences can be found as follows: human thrombin—Genbank Gene ID 2147; bovine thrombin—Genbank Gene ID 280685. In an exemplary embodiment, the thrombin-like enzyme has at least 90%, 91%, 92%, 93%, 94%, 95%, 96%, 97%, 98% or 99% identity with the sequence of Genbank Gene ID 2147. In an exemplary embodiment, the thrombin-like enzyme has at least 90%, 91%, 92%, 93%, 94%, 95%, 96%, 97%, 98% or 99% identity with the sequence of Genbank Gene ID 280685.

DNA inhibitor of the thrombin-like enzyme: A DNA inhibitor of the thrombin-like enzyme inhibits the cleavage of the fibrinogen by the thrombin-like enzyme. A DNA inhibitor of the thrombin-like enzyme can increase by at least 5% the time to a 10% reduction in transmitted light intensity in a Light Scattering Intensity assay (such as described herein), where fibrinogen is used at 10 mg/ml, thrombin is used at between 0.1 and 10 U/ml, and the DNA inhibitor of the thrombin-like enzyme is used at up to 1 M concentration.

In an exemplary embodiment, the DNA inhibitor of the thrombin-like enzyme is itelo. Itelo is a DNA aptamer which can inhibit thrombin. The Itelo oligo can be purchased from Stanford Protein and Nucleic Acid facility (Palo Alto, California, USA) or Integrated DNA Technologies (IDT), using the sequence provided in Tian 2016. The DNA sequence for itelo is: GGTTGGTGTGGTTGGGGGTTAGGGTTAGGGTTAGGGAGTCCGTGGTAGGG CAGGTTGGGGTGACT. In an exemplary embodiment, the DNA inhibitor of the thrombin-like enzyme has at least 90%, 91%, 92%, 93%, 94%, 95%, 96%, 97%, 98% or 99% identity with the DNA sequence of itelo. In an exemplary embodiment, the DNA inhibitor of the thrombin-like enzyme comprises the DNA aptamer TBA. The DNA sequence for TBA is: GGTTGGTGTGGTTGG. In an exemplary embodiment, the DNA inhibitor of the thrombin-like enzyme has at least 90%, 91%, 92%, 93%, 94%, 95%, 96%, 97%, 98% or 99% identity with the DNA inhibitor of the thrombin-like enzyme which comprises the DNA aptamer TBA. In an exemplary embodiment, the DNA inhibitor of the thrombin-like enzyme comprises the DNA aptamer HD22. The DNA sequence for HD22 is: AGTCCGTGGTAGGGCAGGTTGGGGTGACT. In an exemplary embodiment, the DNA inhibitor of the thrombin-like enzyme has at least 90%, 91%, 92%, 93%, 94%, 95%, 96%, 97%, 98% or 99% identity with the DNA inhibitor of the thrombin-like enzyme which comprises the DNA aptamer HD22.

Additional examples of DNA inhibitors of thrombin-like enzymes can be found in Tian et al. (2016) J Am Chem Soc 138 (3): 955; Kim et al. Biochemistry (2009) 106 (16) 6489-6494; Mo et al. Bioconjugate Chem. 2019, 30, 1, 231-241; and Ali et al, Chem. Commun., 2019, 55, 5627-5630, which are incorporated by reference herein for all purposes.

Photoswitchable inhibitor of the DNA inhibitor of the thrombin-like enzyme: These are molecules that reduce the inhibition of thrombin by a DNA inhibitor referenced herein in one state (such as a first conformational isomerization state) compared to another state (such as a second conformational isomerization state), where the change from the first to the second state can be caused by light of a first wavelength, and the change from the second state to the first state either occurs spontaneously or can by caused by light of a distinct second wavelength.

In an exemplary embodiment, the photoswitchable inhibitor of the DNA inhibitor of the thrombin-like enzyme is razo. Razo is an azobenzene derivative which has the following conformational isomers:

trans-Razo:

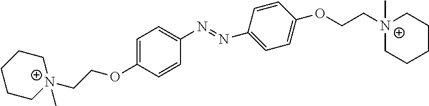 [SMILES: C[N+]1(CCOc2ccc(/N=N/c3ccc(OCC[N+]4(C)CCCCC4)cc3)cc2)CCCCC1]

cis-Razo:

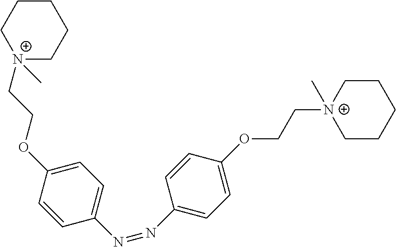 [SMILES: C[N+]1(CCOc2ccc(/N=N\c3ccc(OCC[N+]4(C)CCCCC4)cc3)cc2)CCCCC1]

When in the trans isomer, razo can bind itelo and thus prevent itelo from inhibiting thrombin. When in the cis isomer, razo cannot bind itelo, and thus itelo can inhibit thrombin. Razo was synthesized according to Wang, et al. Angew. Chem., Int. Ed. 2010, 49, 5305). In an exemplary embodiment, the photoswitchable inhibitor of the DNA inhibitor of the thrombin-like enzyme is one that inhibits itelo. In an exemplary embodiment, the photoswitchable inhibitor of the DNA inhibitor of the thrombin-like enzyme is one that inhibits a DNA inhibitor of the thrombin-like enzyme comprising the DNA aptamer TBA. In an exemplary embodiment, the photoswitchable inhibitor of the DNA inhibitor of the thrombin-like enzyme is one that inhibits a DNA inhibitor of the thrombin-like enzyme comprising the DNA aptamer HD22. In an exemplary embodiment, the photoswitchable inhibitor of the DNA inhibitor of the thrombin-like enzyme is one that inhibits a DNA inhibitor of the thrombin-like enzyme comprising the DNA aptamer TBA and the DNA aptamer HD22.

Additional examples of photoswitchable inhibitors of the DNA inhibitors of thrombin-like enzymes can be found in Tian et al. (2016) J Am Chem Soc 138 (3): 955; Kim et al. Biochemistry (2009) 106 (16) 6489-6494; Mo et al. Bioconjugate Chem. 2019, 30, 1, 231-241; and Ali et al, Chem. Commun., 2019, 55, 5627-5630, which are incorporated by reference herein for all purposes.

In an exemplary embodiment, prior to the administering of step a), the thrombin-like enzyme is present in the solution at a concentration of between about 0.125 U/mL and about 2.5 U/mL. In an exemplary embodiment, prior to the administering of step a), the thrombin-like enzyme is present in the solution at a concentration of between about 0.25 U/mL and about 1 U/mL.

In an exemplary embodiment, prior to the administering of step a), the thrombin is present in the solution at a concentration of between about 0.125 U/mL and about 2.5 U/mL. In an exemplary embodiment, prior to the administering of step a), the thrombin is present in the solution at a concentration of between about 0.25 U/mL and about 1 U/mL.

In an exemplary embodiment, prior to the administering of step a), the DNA inhibitor of the thrombin-like enzyme is present in the solution at a concentration of between about 20 nM and about 20 μM. In an exemplary embodiment, prior to the administering of step a), the DNA inhibitor of the thrombin-like enzyme is present in the solution at a concentration of between about 100 nM and about 1 μM.

In an exemplary embodiment, prior to the administering of step a), the itelo is present in the solution at a concentration of between about 20 nM and about 20 μM. In an exemplary embodiment, prior to the administering of step a), the itelo is present in the solution at a concentration of between about 100 nM and about 1 μM.

In an exemplary embodiment, prior to the administering of step a), the photoswitchable inhibitor of the DNA inhibitor of the thrombin-like enzyme is present in the solution at a concentration of between about 10 μM and about 1 mM. In an exemplary embodiment, prior to the administering of step a), the photoswitchable inhibitor of the DNA inhibitor of the thrombin-like enzyme is present in the solution at a concentration of between about 50 μM and about 200 μM.

In an exemplary embodiment, prior to the administering of step a), the razo is present in the solution at a concentration of between about 10 μM and about 1 mM. In an exemplary embodiment, prior to the administering of step a), the razo is present in the solution at a concentration of between about 50 μM and about 200 μM.

### IId3. Buffer

In an exemplary embodiment, the solution further comprises a buffer. In an exemplary embodiment, the buffer has a pH which is between about 5 and about 10. In an exemplary embodiment, the buffer has a pH which is between about 6.5 and about 8.5. In an exemplary embodiment, the buffer has a pH which is between about 7 and about 8. In an exemplary embodiment, the buffer has a pH which is between about 7.2 and about 7.6. In an exemplary embodiment, the buffer is ADA, PIPES, ACES, MOPSO, cholamine chloride, MOPS, BES, TES, DIPSO, TAPSO, acetamidoglycine, POPSO, HEPPSO, HEPPS, tricine, tris, glycinamide, glycylglycine, bicine, and TAPS. In an exemplary embodiment, the buffer is phosphate buffered saline, sodium bicarbonate, HEPES, or MES. In an exemplary embodiment, the buffer is phosphate buffered saline. In an exemplary embodiment, the buffer concentration is between about 0.1× and about 5×. In an exemplary embodiment, the buffer concentration is between about 0.5× and about 2×. In an exemplary embodiment, the phosphate-buffered saline concentration is between about 0.1× and about 5×. In an exemplary embodiment, the phosphate-buffered saline concentration is between about 0.5× and about 2×.

### IId4. Potassium

In an exemplary embodiment, the solution further comprises potassium. In an exemplary embodiment, the solution further comprises potassium phosphate or potassium chloride. In an exemplary embodiment, the potassium is present in a concentration of between about 0.1 mM and about 80 mM. In an exemplary embodiment, the potassium is present in a concentration of between about 3.5 mM and about 5 mM.

### IId5. Calcium

In an exemplary embodiment, the solution further comprises calcium. In an exemplary embodiment, the solution further comprises calcium phosphate or calcium chloride. In an exemplary embodiment, the calcium is present in a concentration of between about 0.1 mM and about 80 mM. In an exemplary embodiment, the calcium is present in a concentration of between about 2.2 mM and about 2.4 mM.

### IId6. Sodium

In an exemplary embodiment, the solution further comprises sodium. In an exemplary embodiment, the solution further comprises sodium phosphate or sodium chloride. In an exemplary embodiment, the sodium is present in a concentration of between about 0.1 mM and about 200 mM. In an exemplary embodiment, the sodium is present in a concentration of between about 100 mM and about 150 mM. In an exemplary embodiment, the sodium is present in a concentration of between about 140 mM and about 160 mM.

### IId7. Mammalian Cells

In an exemplary embodiment, the solution further comprises mammalian cells. In an exemplary embodiment, the solution further comprises human cells. In an exemplary embodiment, the solution further comprises fibroblasts. In an exemplary embodiment, the solution further comprises mammalian fibroblasts. In an exemplary embodiment, the solution further comprises human fibroblasts. In an exemplary embodiment, the solution further comprises human dermal fibroblasts. In an exemplary embodiment, the solution further comprises human neonatal dermal fibroblasts (HNDF). In an exemplary embodiment, the solution further comprises human ventricular cardiac fibroblasts (HNDF). Fibroblasts can be purchased from commercial suppliers such as Lonza of ATCC. Fibroblasts can also be produced from induced pluripotent stem cell (iPSCs) lines, such as SCVI-15 (available through the Stanford Cardiovascular Institute (SCVI biobank). In an exemplary embodiment the fibroblasts are present in the solution at a concentration of between about 0.1 million cells/mL and about 100 million cells/mL. In an exemplary embodiment the fibroblasts are present in the solution at a concentration of between about 0.5 million cells/mL and about 10 million cells/mL.

In an exemplary embodiment, the solution further comprises mammalian cardiomyocytes. In an exemplary embodiment, the solution further comprises human cardiomyocytes. In an exemplary embodiment, the solution further comprises stem cell derived cardiomyocytes. Stem cell derived cardiomyocytes can be produced according to procedures in Ho et al., Adv. Healthcare Mater. 2022, 2201138. In an exemplary embodiment the cardiomyocytes are present in the solution at a concentration of between about 0.1 million cells/mL and about 100 million cells/mL. In an exemplary embodiment the cardiomyocytes are present in the solution at a concentration of between about 2.5 million cells/mL and about 50 million cells/mL.

In an exemplary embodiment, the solution further comprises mammalian endothelial cells. In an exemplary embodiment, the solution further comprises human endothelial cells. In an exemplary embodiment, the solution further comprises human umbilical vein endothelial cells. In an exemplary embodiment, the solution further comprises stem cell derived endothelial cells. Stem cell derived endothelial cells can be produced according to procedures in Ho et al., Adv. Healthcare Mater. 2022, 2201138. Endothelial cells can be purchased from commercial suppliers such as Lonza. In an exemplary embodiment the endothelial cells are present in the solution at a concentration of between about 0.1 million cells/mL and about 100 million cells/mL. In an exemplary embodiment the endothelial cells are present in the solution at a concentration of between about 5 million cells/mL and about 15 million cells/mL.

### IId8. Exemplary Solution Embodiments

In an exemplary embodiment, the solution comprises a first biopolymer subunit, a photoswitchable thrombin-like enzyme system, a buffer, potassium, and human cells. In another exemplary embodiment, the solution comprises fibrinogen, thrombin, itelo, razo, phosphate buffered saline, potassium, and human fibroblasts. In another exemplary embodiment, the solution comprises fibrinogen, thrombin, itelo, razo, phosphate buffered saline, potassium, and human cardiomyocytes. In another exemplary embodiment, the solution comprises fibrinogen, thrombin, itelo, razo, phosphate buffered saline, potassium, and human endothelial cells. In another exemplary embodiment, the solution comprises fibrinogen, thrombin, itelo, razo, phosphate buffered saline, potassium, calcium and human fibroblasts or human cardiomyocytes or human endothelial cells.

In an exemplary embodiment, the solution comprises fibrinogen at a concentration of between about 3 mg/mL and about 100 mg/mL; thrombin at a concentration of between about 0.125 U/mL and about 2.5 U/mL; itelo at a concentration of between about 20 nM and about 20 M; and razo at a concentration of between about 10 μM and about 1 mM. In an exemplary embodiment, the solution comprises fibrinogen at a concentration of between about 3 mg/mL and about 100 mg/mL; thrombin at a concentration of between about 0.125 U/mL and about 2.5 U/mL; itelo at a concentration of between about 20 nM and about 20 M; and razo at a concentration of between about 10 UM and about 1 mM, phosphate buffered saline, potassium, and human fibroblasts.

In an exemplary embodiment, the solution comprises fibrinogen at a concentration of between about 10 mg/mL and about 50 mg/mL; thrombin at a concentration of between about 0.25 U/mL and about 1 U/mL; itelo at a concentration of between about 100 nM and about 1 μM; and razo at a concentration of between about 50 μM and about 200 μM. In an exemplary embodiment, the solution comprises fibrinogen at a concentration of between about 10 mg/mL and about 50 mg/mL; thrombin at a concentration of between about 0.25 U/mL and about 1 U/mL; itelo at a concentration of between about 100 nM and about 1 μM; and razo at a concentration of between about 50 μM and about 200 μM, phosphate buffered saline, potassium, and human fibroblasts and/or human cardiomyocytes and/or human endothelial cells.

IIe. Light of a Second Wavelength

In an exemplary embodiment, the light of a second wavelength inhibits the thrombin-like enzyme of the photoswitchable thrombin-like enzyme system. In an exemplary embodiment, the light of a second wavelength is ultraviolet light. In an exemplary embodiment, the light of a second wavelength is between about 320 nm and about 400 nm. In an exemplary embodiment, the light of a second wavelength is between about 346 nm and about 374 nm. In an exemplary embodiment, the second wavelength is administered between about 0.1 seconds and about 20 seconds. In an exemplary embodiment, the second wavelength is administered between about 0.1 seconds and about 10 seconds. In an exemplary embodiment, the second wavelength is administered between about 0.5 seconds and about 5 seconds. In an exemplary embodiment, the solution receives a second wavelength intensity of between about 10 mW/cm² and about 14 mW/cm². In an exemplary embodiment, the solution receives a second wavelength intensity of between about 6 mW/cm² and about 18 mW/cm².

Illumination of the solution for the light of a first wavelength and/or light of a second wavelength can be provided by a Lumencor Aura or Spectra light engine via a liquid light guide (LLG) optionally capped by a collimator. In an exemplary embodiment, the wavelengths and power output for UV light is 360 nm center wavelength with 28 nm bandpass filter. In an exemplary embodiment, the power output is 300 mW. In an exemplary embodiment, the wavelengths and power output for blue light is 475 nm center wavelength with 28 nm bandpass filter. In an exemplary embodiment, the power output is 500 mW. In an exemplary embodiment, the light is output as a collimated beam of approximately 1 cm in radius: utilizing the power outputs described herein correspond to output intensities of roughly 96 mW/cm² and 159 mW/cm² for UV and blue light, respectively. In an exemplary embodiment, the intensity of blue light into the solution is measured as 12.9% of the intensity at the light source, or 20.5 mW/cm² when the light source is at 100% power. In an exemplary embodiment, the intensity of UV light into the solution is measured as 12.3% of the intensity at the light source, or 11.8 mW/cm² when the light source is at 100% power.

IIf. Repeating

In an exemplary embodiment, the steps a) and b) are repeated as necessary, thereby forming the fibrin mesh. In an exemplary embodiment, the steps a) and b) are repeated until the regions illuminated by the activating wavelength have formed the fibrin mesh but the regions illuminated by the inhibiting wavelength have not. In an exemplary embodiment, the steps a) and b) are repeated until the regions illuminated by the light of a first wavelength have formed the fibrin mesh but the regions illuminated by the light of a second wavelength have not. In an exemplary embodiment, the steps a) and b) are repeated for between about 10 seconds and about 90 minutes. In an exemplary embodiment, the steps a) and b) are repeated for between about 10 seconds and about 60 minutes. In an exemplary embodiment, the steps a) and b) are repeated for between about 10 minutes and about 60 minutes. In an exemplary embodiment, the steps a) and b) are repeated for between about 10 minutes and about 30 minutes. In an exemplary embodiment, the steps a) and b) are repeated for between about 10 seconds and about 10 minutes. In an exemplary embodiment, the steps a) and b) are repeated for between about 1 minute and about 10 minutes. In an exemplary embodiment, the steps a) and b) are repeated for between about 1 minute and about 5 minutes. In an exemplary embodiment, the steps a) and b) are repeated for between about 5 minutes and about 10 minutes. In an exemplary embodiment, the steps a) and b) are repeated for between about 2 minutes and about 4 minutes. In an exemplary embodiment, the steps a) and b) are repeated for between about 2 minutes and about 5 minutes. In an exemplary embodiment, the steps a) and b) are repeated between about 5 times seconds and about 2.700 times. In an exemplary embodiment, the steps a) and b) are repeated between about 5 times and about 1.800 times. In an exemplary embodiment, the steps a) and b) are repeated between about 300 times and about 1.800 times. In an exemplary embodiment, the steps a) and b) are repeated between about 300 times and about 900 times. In an exemplary embodiment, the steps a) and b) are repeated between about 5 times and about 300 times. In an exemplary embodiment, the steps a) and b) are repeated between about 30 times and about 300 times. In an exemplary embodiment, the steps a) and b) are repeated between about 30 times and about 150 times. In an exemplary embodiment, the steps a) and b) are repeated between about 150 times and about 300 times. In an exemplary embodiment, the steps a) and b) are repeated between about 60 times and about 120 times. In an exemplary embodiment, the steps a) and b) are repeated between about 60 times and about 150 times.

IIg. Washing

In an exemplary embodiment, the method further comprises d) washing the product of step c) with a washing buffer, thereby forming the fibrin mesh. In an exemplary embodiment, the washing removes the liquid portion of the solution away from the fibrin mesh. In an exemplary embodiment, the washing removes the liquid portion of the solution away from the fibrin mesh. In an exemplary embodiment, the washing buffer is a buffer described herein. In an exemplary embodiment, the washing buffer is phosphate-buffered saline. In an exemplary embodiment, the product of step c) is washed with a washing buffer in an amount which is between 1× and 20× the solution volume. In an exemplary embodiment, the product of step c) is washed with a washing buffer in an amount which is between 1× and 10× the solution volume. In an exemplary embodiment, the product of step c) is washed with a washing buffer in an amount which is between 1× and 5× the solution volume. In an exemplary embodiment, the product of step c) is washed with a washing buffer in an amount which is between 1× and 3× the solution volume. In an exemplary embodiment, the product of step c) is washed with a washing buffer in an amount which is between 2× and 4× the solution volume.

IIh. Exemplary Method Combinations

In an exemplary embodiment, the invention is a method of making a mammalian cell-containing fibrin mesh, comprising: a) administering a pattern of light of a first wavelength of between about 420 nm and about 500 nm into a solution comprising fibrinogen, thrombin, itelo, razo, potassium, calcium, phosphate buffered saline and mammalian cells selected from the group consisting of human fibroblasts, human cardiomyocytes, and human endothelial cells, thereby activating the thrombin; b) administering a pattern of light of a second wavelength of between about 320 nm and about 400 nm into the solution, thereby inhibiting the thrombin; c) repeating a) and b) as necessary, thereby forming the mammalian cell-containing fibrin mesh. In an exemplary embodiment, the mammalian cell-containing fibrin mesh is not otherwise modified with photocrosslinkable moieties.

In an exemplary embodiment, the invention is a method of making a mammalian cell-containing fibrin mesh, comprising: a) administering light of a first wavelength of between about 420 nm and about 500 nm into a solution comprising fibrinogen, thrombin, itelo, razo, potassium, calcium, phosphate buffered saline and mammalian cells selected from the group consisting of human fibroblasts, human cardiomyocytes, and human endothelial cells, thereby activating the thrombin; b) administering light of a second wavelength of between about 320 nm and about 400 nm into the solution, thereby inhibiting the thrombin; c) repeating a) and b) as necessary, thereby forming the mammalian cell-containing fibrin mesh. In an exemplary embodiment, the mammalian cell-containing fibrin mesh is not otherwise modified with photocrosslinkable moieties. In an exemplary embodiment, step c) is repeated as mentioned in Section IIf. In an exemplary embodiment, the method of this paragraph further comprises d) washing the product of step c) with a washing buffer. In an exemplary embodiment, step d) is as mentioned in Section IIg.

In an exemplary embodiment, the invention is a method of making a mammalian fibroblast-containing fibrin mesh, comprising: a) administering light of a first wavelength of between about 420 nm and about 500 nm into a solution comprising mammalian fibroblasts, fibrinogen, thrombin, itelo, razo, potassium, calcium, and phosphate buffered saline, thereby activating the thrombin; b) administering light of a second wavelength of between about 320 nm and about 400 nm into the solution, thereby inhibiting the thrombin; c) repeating a) and b) as necessary, thereby forming the mammalian fibroblast-containing fibrin mesh. In an exemplary embodiment, the fibroblast-containing fibrin mesh is not otherwise modified with photocrosslinkable moieties. In an exemplary embodiment, the photocrosslinkable moieties are acrylates, vinylesters, expoxy, and/or thiolenes. In an exemplary embodiment, step c) is repeated as mentioned in Section IIf. In an exemplary embodiment, the method of this paragraph further comprises d) washing the product of step c) with a washing buffer. In an exemplary embodiment, step d) is as mentioned in Section IIg.

In an exemplary embodiment, the invention is a method of making a mammalian myocardiocyte-containing fibrin mesh, comprising: a) administering light of a first wavelength of between about 420 nm and about 500 nm into a solution comprising mammalian myocardiocytes, fibrinogen, thrombin, itelo, razo, potassium, calcium, and phosphate buffered saline, thereby activating the thrombin; b) administering light of a second wavelength of between about 320 nm and about 400 nm into the solution, thereby inhibiting the thrombin; c) repeating a) and b) as necessary, thereby forming the mammalian myocardiocyte-containing fibrin mesh. In an exemplary embodiment, the fibroblast-containing fibrin mesh is not otherwise modified with photocrosslinkable moieties. In an exemplary embodiment, the photocrosslinkable moieties are acrylates, vinylesters, expoxy, and/or thiolenes. In an exemplary embodiment, step c) is repeated as mentioned in Section IIf. In an exemplary embodiment, the method of this paragraph further comprises d) washing the product of step c) with a washing buffer. In an exemplary embodiment, step d) is as mentioned in Section IIg.

In an exemplary embodiment, the invention is a method of making a mammalian endothelial cell-containing fibrin mesh, comprising: a) administering a pattern of light of a first wavelength of between about 420 nm and about 500 nm into a solution comprising mammalian endothelial cells, fibrinogen, thrombin, itelo, razo, potassium, calcium, and phosphate buffered saline, thereby activating the thrombin: b) administering a pattern of light of a second wavelength of between about 320 nm and about 400 nm into the solution, thereby inhibiting the thrombin; c) repeating a) and b) as necessary, thereby forming the mammalian endothelial cell-containing fibrin mesh. In an exemplary embodiment, the fibroblast-containing fibrin mesh is not otherwise modified with photocrosslinkable moieties. In an exemplary embodiment, the photocrosslinkable moieties are acrylates, vinylesters, expoxy, and/or thiolenes. In an exemplary embodiment, step c) is repeated as mentioned in Section IIf. In an exemplary embodiment, the method of this paragraph further comprises d) washing the product of step c) with a washing buffer. In an exemplary embodiment, step d) is as mentioned in Section IIg.

### III. Product-by-Process

In an exemplary embodiment, the invention is a composition produced by a method described herein. In an exemplary embodiment, the invention is a fibrin mesh produced by a method described herein. In an exemplary embodiment, the invention is a fibrin mesh described herein produced by a method described herein. In an exemplary embodiment, the fibrin mesh described herein produced by a method described herein is essentially free of gelatin methacryloyl or polyethylene glycol diacrylate. In an exemplary embodiment, between about 0.01% and about 5%, or between about 0.01% and about 4%, or between about 0.01% and about 3%, or between about 0.01% and about 3%, or between about 0.01% and about 2%, or between about 0.01% and about 1%, or between about 0.01% and about 0.5% of the volume of the fibrin mesh described herein produced by a method described herein contains gelatin methacryloyl or polyethylene glycol diacrylate. In an exemplary embodiment, between about 0.01% and about 5%, or between about 0.01% and about 4%, or between about 0.01% and about 3%, or between about 0.01% and about 3%, or between about 0.01% and about 2%, or between about 0.01% and about 1%, or between about 0.01% and about 0.5% of the weight of the fibrin mesh described herein produced by a method described herein contains gelatin methacryloyl or polyethylene glycol diacrylate.

In an exemplary embodiment, the terms ‘sample’ and ‘solution’ are used interchangeably.

It is to be understood that the present invention covers all combinations of aspects and/or embodiments, as well as suitable, convenient and preferred groups described herein.

The invention is further illustrated by the Examples that follow. The Examples are not intended to define or limit the scope of the invention.

### EXAMPLES

### Example 1

### Assessment of Razo-Itelo System Clotting Kinetics

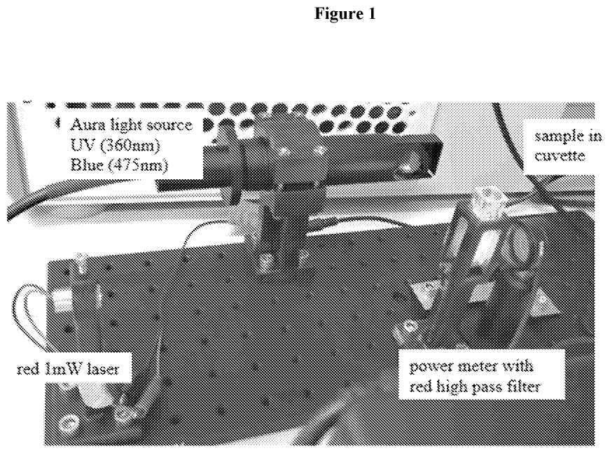

Light scattering was used to measure the progression of the clotting reaction, where as the clot forms (soluble fibrinogen turns into insoluble fibrin which forms fibrils) it scatters more light. The reaction mixture is placed in a cuvette and a low-power red laser is shown through the cuvette. A power meter behind a 610 nm high-pass filter monitors the amount of red light that is transmitted through the cuvette, and the transmitted light power decreases as the solution clots. See FIG. 1.

The control “Thrombin only” (black dashed line) sample consists of buffer (20 mM Tris-HCl, pH 7.4, 40 mM KCl), 6 mg/ml fibrinogen, and 0.5 U/ml thrombin. The experimental samples (blue and purple curves) consist of the same plus 400 nM Itelo (DNA aptamer; sequence provided Tian et al. (2016) J Am Chem Soc 138 (3): 955) and 600 μM Razo (azo benzene derivative; Razo is synthesized according to a previously reported procedure. (Wang, et al. Angew. Chem., Int. Ed. 2010, 49, 5305). For the UV sample the sample was exposed to constant UV light and for the Blue sample the sample was exposed to constant Blue light, each maintaining Razo in the maximum cis and maximum trans for UV and Blue light, respectively.

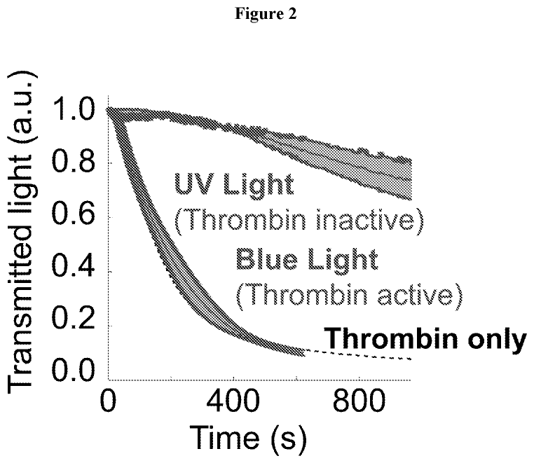

An alternate solution of the invention has the following composition: Buffer (1× PBS, pH 7.4): 2.5 mM Calcium Chloride: 15 mg/ml fibrinogen: 0.5 U/ml thrombin: 100-400 nM Itelo; and 200 μM Razo. See FIG. 2.

### Example 2

### Razo Isomerization Light Kinetics, Dark Kinetics, Cyclic Durability

Razo as a stock solution of 70 mM in DMSO was diluted to 25 μM Razo in buffer (20 mM Tris-HCl, pH 7.4, 40 mM KCl). Absorbance spectra were acquired on a PerkinElmer UV-Vis spectrometer and sample illumination light was from a Lumencor Aura light engine, with a Blue light output centered on 475 nm with 28 nm bandpass filter and UV light output centered on 360 nm with a 28 nm bandpass filter. cis-Razo and trans-Razo Absorbance spectra shapes agreed with those from the paper where Razo was originally developed (Wang, et al. Angew. Chem., Int. Ed. 2010, 49, 5305). We aimed to characterize several features of the isomerization that are relevant to inform printing parameters for patterning of the substrate: light kinetics, dark kinetics, and durability.

### Conditions for Razo Cis-Trans Conversion Using Light

After a long Blue (referred to as ‘visible’ in legend) light exposure to drive Razo to a trans dominated photostationary state, the amount of UV light necessary to drive Razo to the cis dominated photostationary state was determined to be roughly 10 seconds at 25% power (approximately 15 mW/cm²).

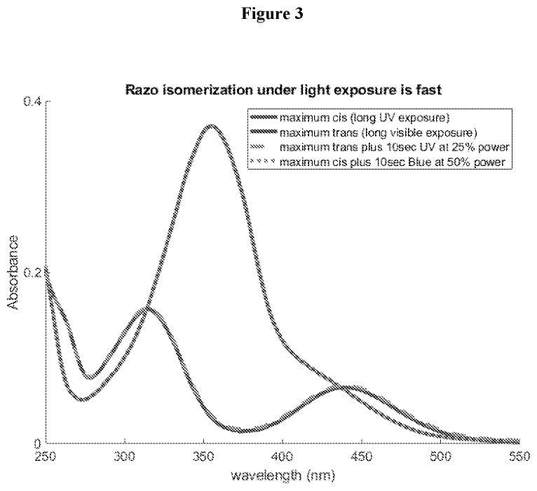

After a long UV light exposure to drive Razo to a cis dominated photostationary state, the amount of Blue light necessary to drive Razo to the trans dominated photostationary state was determined to be roughly 10 seconds at 50% power (approximately 50 mW/cm²). See FIG. 3.

### Conditions for Razo Cis-Trans Thermal Conversion (in the Dark)

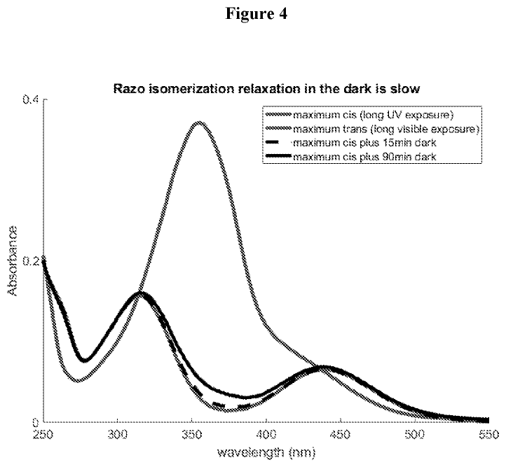

After a long UV exposure to drive Razo to the cis dominated photostationary state, the sample was kept in the dark for 15 and 90 minutes. There was almost no relaxation of cis to trans after 15 minutes, suggesting that relaxation at the shorter time scales relevant for printing is negligible. See FIG. 4.

### Durability of the Razo Cis-Trans Interconversion

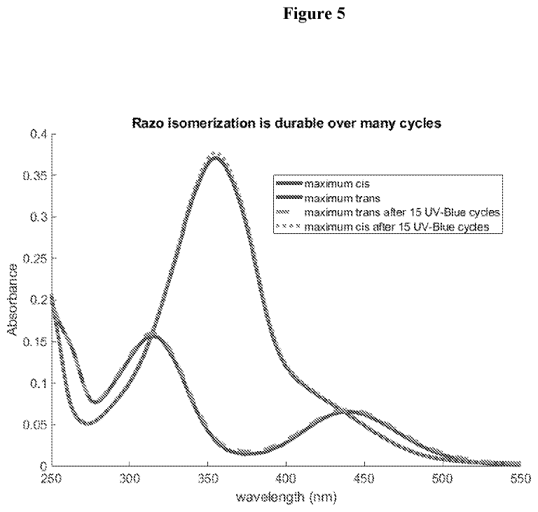

Blue light was applied until the trans-dominated photostationary state was reached (blue curve in FIG. 5). Then UV light was applied until the cis-dominated photostationary state was reached (purple curve in FIG. 5). This process was repeated 15 times and the spectra at each photostationary state were remeasured (gray dashed lines), showing that isomerization efficiency is maintained over 15 cycles. This suggests that repeated application of blue then UV light could be used to cycle thrombin as part of a multi-exposure or multi-layer 3D printing system.

### Example 3

### Printing in Inverted Stereolithography Setup

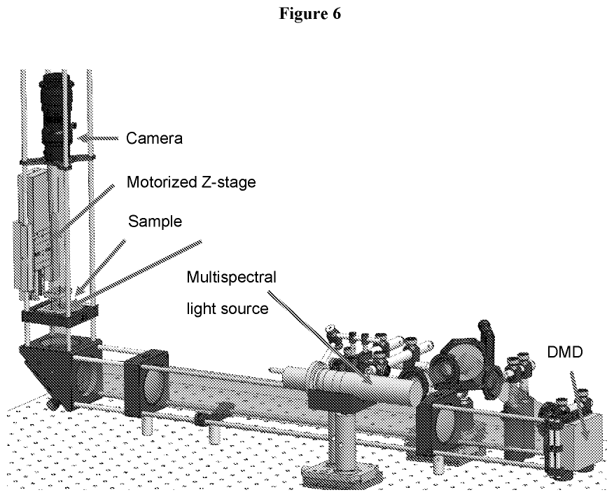

The inverted stereolithography setup was constructed using a Digital Micromirror Device (DMD) from DLi, near-UV achromatic lenses from Edmund Optics, and optomechanical components from ThorLabs. The image created by the DMD is projected onto the polymerization plane by the lenses. The polymerization plane is at the bottom of a vat of the sample, which is—aside from the optional addition of mammalian cells-similar to the Example 1 composition: buffer (20 mM Tris-HCl, pH 7.4, 40 mM KCl), 6 mg/ml fibrinogen, 0.5 U/ml thrombin, 400 nM Itelo, and 600 μM Razo. See FIG. 6.

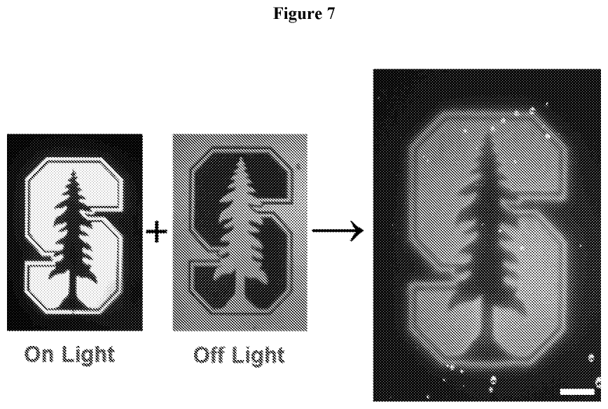

To form an image, a blue light pattern (on light) is applied to the polymerization plane, and the inverse pattern (off light) is applied with UV light to the polymerization plane. The light source is synced with the DMD such that the color and patterns alternate in sync. For example, the Stanford logo was printed as an opaque fibrin mesh. Scale bar: 1 mm. See FIG. 7.

The resulting opaque fibrin mesh was imaged using dark-field microscopy, wherein collimated green light from the Aura light source was applied to the glass slide on which the scaffold pattern was produced. Thus, light that is scattered by the fibrin mesh is detected at the camera and appears green, while the unpolymerized and transparent fibrinogen solution appears dark.

### Resolution of the Fibrin Mesh

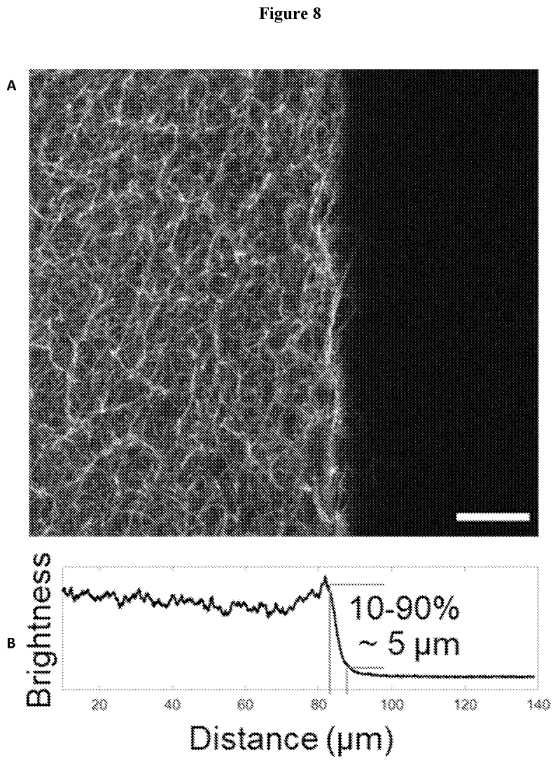

Another fibrin mesh scaffold was prepared in the same manner as described above. After fibrin had polymerized (post-printing) the unpolymerized fibrinogen was washed away by gentle submersion in phosphate buffered saline (PBS). Next, the polymerized fibrin mesh was visualized under high resolution fluorescence microscopy by using NHS-fluorescein to label the primary amine groups on fibrin post-printing. The image is a maximum intensity z-projection captured using fluorescence confocal microscopy. The crispness of the printed edge illustrates the contrast of the clotted fibrin region from the adjacent (i.e. absence of fibrin) region. Scale bar: 20 μm. Critically, observed is a natural, porous, fibrillar structure of the printed fibrin. The natural fibrin material, micrometer-scale pores, and fibrillar structure are advantageous for encapsulated cells (FIG. 8B). Materials used in the prior art entomb encapsulated cells in gels with synthetic components and nanometer-scale pores which are often non-degradable. See FIG. 8.

### Example 4

### Fibroblasts and Fibrin Mesh

The viability of fibroblasts placed on the fibrin mesh was ascertained. Primary fibroblasts were purchased from Lonza Inc., and cultured in tissue culture flasks containing Dulbecco's Modified Eagle Medium (DMEM, Thermo Fisher Scientific) supplemented with 10% fetal bovine serum (FBS, Thermo Fisher Scientific) and 100 U/ml penicillin/streptomycin (Thermo Fisher Scientific). After the fibroblasts reached confluency, they were lifted off via application of trypsin-EDTA for 3 minutes, followed by washing in DMEM+10% FBS, centrifugation at 300×g for 5 minutes, and resuspended in fresh DMEM+10% FBS. These fibroblasts were then added to the solution, exposed to light as described in Example 3, and subsequently rinsed in DMEM+10% FBS medium to remove unpolymerized fibrinogen.

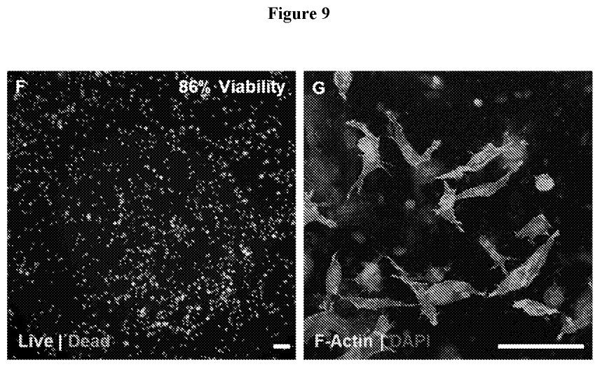

Fibroblasts were assayed for viability by mixing in 0.5 μl/min Calcein-AM/and 1 μl/min Ethidium Homodimer-1 Live/DEAD assay (Thermofisher Scientific) into DMEM/F12 media containing 10% FBS and 1% penicillin/streptomycin, and incubated for 30 minutes. The fibroblasts were then imaged in an epifluorescence microscope (Nikon Ts2). Printed constructs with fibroblasts show high viability, demonstrated by a high ratio of calceinAM (green):ethidium homodimer-1 (red) staining (FIG. 9F).

After 36 hours of growth, fibroblasts were imaged using phalloidin-Alexa Fluor 488 (to stain F-actin) and DAPI (to stain nuclei) to visualize cell morphology (FIG. 9G). The F-actin staining illustrates migratory cell behavior, indicated by the non-spherical nature of the fibroblasts, their elongated shape, and their extensive lamellipodia and filopodia. This indicates a high degree of cell adhesion, cell compatibility, and biodegradability of the photopatterned fibrin mesh.

### Example 5

### Multi-Layer Patterned Substrate

The inverted stereolithography setup described in Example 3 can be utilized to generate a multi-layer patterned substrate. In such an embodiment, the first layer can be printed as described in Example 3. The first layer can be attached to a flat surface known as the build platform, which is actuated by a linear stage. After the first layer is printed, the build platform can be moved upward a certain distance, and the printed first layer can be moved with it. Then a second layer can be printed underneath the first layer. Then the build platform can be moved upward the same distance, and a third layer can be printed beneath the second layer.

This process can be repeated until the multi-layer patterned substrate has been printed. The illumination patterns can be the same or different for each layer.

### Example 6

Printing and Subsequent Perfusion of Cell-Laden Fibrin with Bifurcated Channel in Fluidic Chip

Human fibrinogen (Sigma F3879 or 341576) was reconstituted at 100 mg/ml in 1× Phosphate Buffered Saline (PBS) without calcium or magnesium. PBS pre-warmed to 37 C was gently added to the fibrinogen powder, followed by incubation for at least one hour at 37 C in a humidified incubator before mixing by gentle trituration. Aliquots were frozen at −80 C. When used, fibrinogen stock solution was after thawing kept at room temperature (as fibrinogen can precipitate at 4 C). Fresh aliquots (no more than several hours post-thaw) were used for all experiments, and aliquots were never re-frozen.

Human thrombin (Sigma T6884) was reconstituted at 25 U/ml in PBS without calcium or magnesium. Aliquots were frozen at −80 C. When used, thrombin stock solution was after thawing kept on ice. Fresh aliquots (no more than several hours post-thaw) were used for all experiments, and aliquots were never re-frozen.

A PDMS chip was fabricated with inlet and outlet needles serving a rectangular well in which the sample was printed. The inlet and outlet needles had luer lock fittings allowing connection to syringes or tubing for flowing in fluids including the sample, wash buffer, and cell culture media.

Separate, equal volume (“half reaction”) solutions of thrombin and fibrinogen were prepared in 1.5 ml Eppendorf tubes. The total reaction volume was 400 ul, with each half reaction being 200 ul. Equal volumes for the half reactions were used as having equal volumes is efficient for mixing. Itelo and Razo were in the thrombin side of the reaction. Fibrinogen pre-labeled with Alexa Fluor 488 or Fluorescein was mixed in at a 1:10 ratio to unlabeled fibrinogen to achieve the specified total fibrinogen concentration. (This method results in a fibrin mesh that is fluorescently labeled without damage to the cells, allowing imaging at multiple time points over multiple days.) Fibroblasts were added to the fibrinogen side as follows: Human Neonatal Dermal Fibroblasts (HNDFs) were cultured in T25 or T75 culture flasks in DMEM+10% fetal bovine serum (FBS)+1% Penicillin-Streptomycin (Pen-Strep), and split every 3-5 days with trypsin. To harvest cells for printing, cells were lifted with trypsin, which was subsequently quenched with media, and resuspended in PBS as a wash. To be able to visualize cells in the fibrin matrix, Cytotracker Red CMTPX (ThermoFisher C34552) was used according to the manufacturer's protocol. Briefly, the dye was dissolved into DMSO to obtain a final stock concentration of 10 mM, the stock concentration was then diluted using serum free medium to a final working concentration of 10 μM, which was then warmed up to 37° C. The harvested cell pellet was resuspended in the CellTracker working solution, followed by a 30 min incubation at 37° C. The CellTracker working solution was removed by centrifugation at 300×g for 5 minutes, followed by washing in PBS, centrifugation at 300×g for 5 minutes, and resuspension in PBS. Cells were then pelleted, as much PBS as possible was aspirated before resuspending in fibrinogen solution at a fibrinogen concentration appropriate for the fibrinogen side of the reaction. Cell concentration was varied in different experiments from 0.5 to approximately 10 million/ml. Final component concentrations were as follows in 1× PBS: fibrinogen (50) mg/ml), thrombin (0.5 U/ml), Itelo (600 nM), Razo (200 μM).

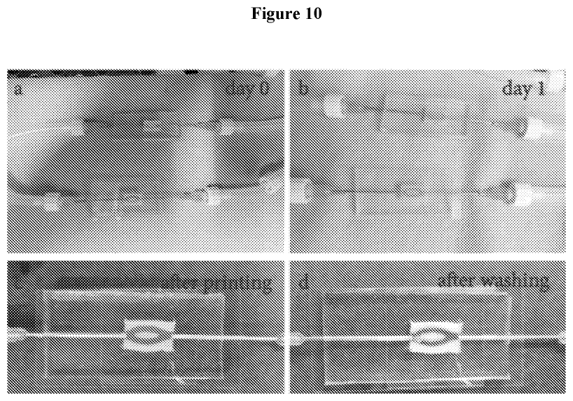

The thrombin half-reaction was first exposed to UV light to set the Razo system to “OFF”. Next, the fibrinogen and thrombin half reactions were quickly, gently, and thoroughly mixed to start the coagulation process. The resulting mixture was then transferred by syringe into the PDMS chip which held the mixture at the focal plane (sample plane) of the stereolithography printer. Two different patterns, one pattern being the inverse of the other, were formed by the DMD and alternated every 1 second without pause. The pattern for the prints shown in the figure was such that fibrin filled the entire chip space except for a bifurcated channel that began from the inlet needle, bifurcated, and then reanastomosed before leading to to the outlet needle. The pattern corresponding to the fibrin area was illuminated with blue light while the pattern corresponding to the channels was illuminated with UV light. The light engine was set to 100% power in both the UV and Blue channels. At a pre-determined (by LSI assay) time point when the blue-illuminated, but not the UV-illuminated, regions had transitioned from liquid to gel, the printing process was stopped and unpolymerized solution was washed away by flowing PBS to leave the polymerized fibrin mesh. (FIG. 10C and FIG. 10D: note the translucent fibrinogen solution before washing and the clear PBS after washing). This time point varied from 1 to 30 minutes depending on the recipe used. For the print shown in the figure, printing was stopped after 12 minutes.

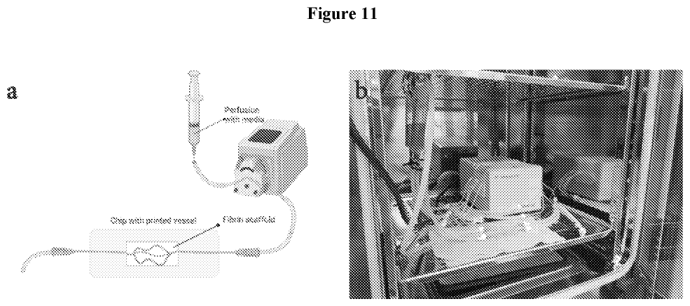

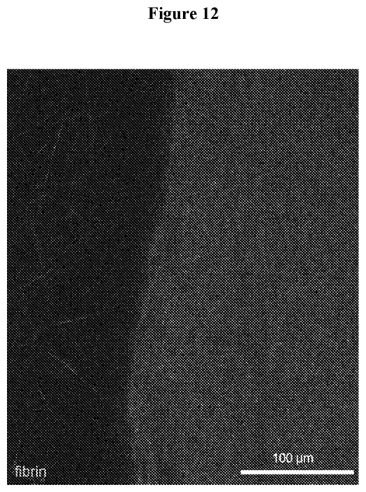

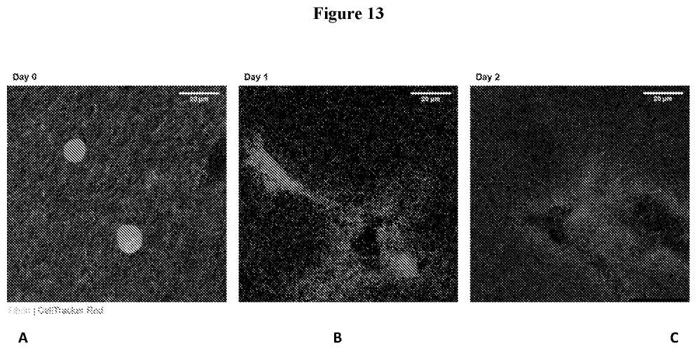

Cell culture media (DMEM+10% fetal bovine serum+1% pen-strep) was perfused continuously via the needles through the chip using an Ismatec pump (FIG. 11A), first displacing the PBS in the channels (FIG. 10A, immediately after perfusion hookup) and then diffusing through the printed fibrin and reaching the cells inside the matrix (FIG. 10B). The media perfusion takes place in a standard, humidified cell culture incubator at 37° C. and 5% CO₂ (FIG. 11B). The fibroblasts were then imaged in an inverted confocal microscope (Leica SP8), the clear channels can be observed at 100× magnification (FIG. 12) and CellTracker labelled HNDF embedded in labeled fibrin were imaged 0 (FIG. 13A), 24 (FIG. 13B) and 48 (FIG. 13C) hours after printing. After 24 hours, fibroblast cells inside the fibrin have elongated and appear to have degraded some of the fibrin matrix around themselves, as is desired.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of making a fibrin mesh, comprising:
a) administering light of a first wavelength into a solution,
wherein the solution comprises fibrinogen and a photoswitchable thrombin-like enzyme system,
wherein the light of a first wavelength results either in activating or inhibiting the thrombin-like enzyme in the photoswitchable thrombin-like enzyme system;
b) administering a light of a second wavelength into the solution, wherein the light of a second wavelength results either in activating or inhibiting the thrombin-like enzyme in the photoswitchable thrombin-like enzyme system;
c) repeating a) and b) as necessary, thereby forming the fibrin mesh,
wherein the fibrin mesh is essentially free of gelatin methacryloyl or polyethylene glycol diacrylate;
wherein the photoswitchable thrombin-like enzyme system comprises a thrombin-like enzyme, a DNA inhibitor of the thrombin-like enzyme, and a photoswitchable 14 inhibitor of the DNA inhibitor of the thrombin-like enzyme;
wherein when the light of a first wavelength activates the thrombin-like enzyme, then the light of a second wavelength inhibits the thrombin-like enzyme; and
wherein when the light of a first wavelength inhibits the thrombin-like enzyme, then the light of a second wavelength activates the thrombin-like enzyme.

2. The method of claim 1, wherein
the light of a first wavelength activates the thrombin-like enzyme in the system, the light of a second wavelength inhibits the thrombin-like enzyme in the system.

3. The method of claim 1, wherein the first wavelength is between about 420 nm and about 500 nm.

4. The method of claim 1, wherein the first wavelength is administered between about 0.5 seconds and about 5 seconds.

5. The method of claim 1, wherein the solution receives a first wavelength intensity of between about 18.5 mW/cm² and about 22.5 mW/cm².

6. The method of claim 1, wherein the second wavelength is between about 320 nm and about 400 nm.

7. The method of claim 1, wherein the second wavelength is administered between about 0.5 seconds and about 5 seconds.

8. The method of claim 1, wherein the solution receives a second wavelength intensity of between about 10 mW/cm² and about 14 mW/cm².

9. The method of claim 1, wherein, prior to the administering of step a), the fibrinogen is present in the solution at a concentration of between about 3 mg/ml and about 100 mg/mL.

10. The method of claim 1, wherein the thrombin-like enzyme is thrombin, the DNA inhibitor of the thrombin-like enzyme is itelo, and the photoswitchable inhibitor of the DNA inhibitor of the thrombin-like enzyme is razo.

11. The method of claim 10, wherein, prior to the administering of step a),
the thrombin is present in the solution at a concentration of between about 0.125 U/mL and about 2.5 U/mL,
the itelo is present in the solution at a concentration of between about 20 nM and about 20 μM, and
the razo is present in the solution at a concentration of between about 10 μM and about 1 mM.

12. The method of claim 1, wherein the solution further comprises mammalian cells.

13. The method of claim 12, wherein the mammalian cells are fibroblasts, cardiomyocytes, or endothelial cells.

14. The method of claim 1, wherein the solution further comprises potassium.

15. The method of claim 14, wherein the potassium is present in a concentration of between about 0.1 mM and about 80 mM.

16. The method of claim 1, wherein the solution further comprises calcium.

17. The method of claim 16, wherein the calcium is present in a concentration of between about 0.1 mM and about 80 mM.

18. The method of claim 1, wherein the solution further comprises a buffer.

19. The method of claim 18, wherein the buffer is phosphate-buffered saline.

20. The method of claim 1, wherein the solution is essentially free of photocrosslinkable moieties.

21. The method of claim 20, wherein the photocrosslinkable moieties are acrylates, epoxides, vinyl esters, or thiolenes.

22. A fibrin mesh produced by the method of claim 1.
